(19) United States
(12) Patent Application Publication
(10) Pub. No.: US 2022/0088186 A1
(43) Pub. Date: Mar. 24, 2022

(54) SAMRNA VACCINE AND PREPARATION METHOD THEREFOR

(71) Applicant: CANSINO BIOLOGICS INC ., Tianjin (CN)

(72) Inventors: Tao ZHU, Tianjin (CN); Junqiang LI, Tianjin (CN); Shoubai CHAO, Tianjin (CN); Chunlin XIN, Tianjin (CN); Wei MIAO, Tianjin (CN); Xishan LU, Tianjin (CN)

(21) Appl. No.: 17/420,608

(22) PCT Filed: Jan. 7, 2020
(86) PCT No.: PCT/CN2020/070734

§ 371 Date: Jul. 2, 2021

(30) Foreign Application Priority Data
Jan. 7, 2019 (CN) 201910010764.1

(51) Int. Cl. A61K 39/25; A61K 39/15; A61K 39/12; A61K 39/39; A61K 39/00; A61P 31/22

(52) U.S. Cl. CPC A61K 39/25; A61K 2039/55522; A61K 39/12; A61K 39/39; A61K 39/001186; A61K 39/001184; A61K 39/001195; A61K 39/001194; A61K 39/001166; A61K 39/001132; A61K 39/001193; A61K 39/001129; A61K 39/001161; A61K 39/001157; A61P 31/22; A61K 2039/53; A61K 2039/5256; A61K 2039/55533; A61K 2039/55538; A61K 2039/55527; A61K 2039/55561; A61K 39/15

(57) ABSTRACT

Disclosed is an SamRNA vaccine, including a recombinant viral vector which includes: i) a viral gene replication complex including nucleotide sequences encoding viral gene replication-related proteins nsP1, nsP2, nsP3, and nsP4; and ii) a nucleotide sequence encoding at least one antigen. According to the SamRNA vaccine of the present invention, in addition to that a promoter of a modified adenoviral vector itself can transcribe an antigen gene to form mRNA, the viral gene replication-related proteins nsP1-4 use RNA as a template to synthesize a large amount of mRNAs, and the immune effect of a target antigen is greatly improved.

### TECHNICAL FIELD

The present invention belongs to the technical field of vaccines, and particularly relates to a process technology for producing an mRNA vaccine by a modified adenoviral vector and a preparation method thereof, and the mRNA vaccine produced by the adenoviral vector can be continuously amplified by using RNA as a template to form more mRNAs, and is called samRNA (self-amplifying mRNA) for short.

### BACKGROUND

An mRNA vaccine is a gene vaccine with immunity, safety and flexibility. The mRNA vaccine can stimulate an immune system to produce balanced and long-term protection. Some mRNA vaccines themselves have the characteristics of a vaccine adjuvant. The mRNA vaccines can stimulate the immune system in many manners such as generating multiple cytokines, so as to enhance the response ability of an immune body, shorten the immune response time and increase the ability of antibody synthesis and release.

In 1990, scientists injected messenger RNA (mRNA) transcribed in vitro into mice, and found that the mRNA could express its activity in the mice, produce related proteins, and have dose-dependence by detection. This method of injecting the mRNA directly can produce an immune response by expressing a specific protein, which is an embryonic form of an mRNA therapy.

Compared with traditional vaccines, the mRNA has more advantages in safety, causes no insertion of gene mutation, and can be degraded by normal cells, and its half-life can be changed by adjusting sequence modification and delivery vectors. More importantly, traditional vaccines are powerless against many new viruses, not to mention cancer, which is a disease which seriously threatens human health. The acting mechanism of the mRNA makes it like a meal menu. As long as the RNA sequences are encoded, cells can be turned into small drug factories, and the mRNA guides the cells to produce specific proteins to exert a systemic pharmaceutical effect by themselves.

An acting mechanism of the mRNA vaccine: the mRNA participates in the intermediate steps of DNA transcription and protein generation. Currently, there are two kinds of RNAs used for making vaccines, i.e., a non-replicating mRNA and a self-amplifying mRNA (SamRNA). A antigen encoded by a traditional mRNA vaccine contains 5′ and 3′ untranslated regions (UTRs), while the self-amplifying mRNA can not only encode an antigen, but also have a sequence similar to a virus replication process, so that it can replicate in cells and increase the expression quantity of the protein.

The mRNA can be formed by transcribing through a cDNA template in vitro, and an open reading frame (ORF) for protein encoding is added into the mRNA in the late transcription period, so that the synthesized mRNA has the function of encoding a protein. The ORF consists of at least two important elements: a “cap” structure at the 5′ terminal and a “tail” of poly A. In addition, untranslated regions (UTRs) and other complexes are increased to help the stable transcription of the mRNA.

However, a naked mRNA will be degraded when entering the body directly, and efficient mRNA delivery is a guarantee for vaccine efficacy, so developing an efficient mRNA delivery vector is a key factor to ensure vaccine effectiveness. Currently, the administration modes of the mRNA are mainly intradermal or intraarticular injection. However, for such an injection mode, due to the degradation of the mRNA in vivo, administration with a large dosage is needed to stimulate the body to produce an effective immune response. It has been reported in literatures that protamine and other polymers, such as liposomes, can be used for encapsulating the mRNA, thereby avoiding a large amount of damages to the mRNA by proteases in the body. Compared with direct injection, this encapsulation method can effectively increase the body absorption of antigens, but there are still some defects to be overcome, such as the effective encapsulation amount, the release time of the encapsulated mRNA in vivo, and the release and transfer of the mRNA in vivo.

Currently, an adenovirus (Ad) vector system has been widely used in the development of gene therapy drugs and vaccines, there are also many reports of the Ad vector system in clinic, and its safety and reliability as a gene delivery vector have been fully proved. According to whether it can replicate, the adenoviral vector is divided into a replication type and a replication-deficient type. Currently, the replication-defective-type adenoviral vector is more commonly used. Compared with the replication-type adenoviral vector, the replication-defective-type adenoviral vector has better safety and lower immunogenicity of the vector itself. An adenoviral vector vaccine refers to that a protective antigen gene is recombined into a virus genome with an adenovirus as a vector. The antigen gene in the adenoviral vector is protected by a virus capsid protein, which can avoid the degradation of the carried gene by various proteases in the host and effectively solve the degradation risk of the mRNA from an injection site to a host cell. After the host cell is infected with the adenoviral vector, the antigen gene or target gene carried by the adenoviral vector can be expressed into a protein.

Besides being easy to degrade and low in bioavailability, the mRNA vaccine also has a problem of copy number. Whether the mRNA is directly injected or a commonly-used protamine-encapsulated mRNA, one mRNA can only be effectively translated once in an organism, which greatly limits the availability of the mRNA as the vaccine. Therefore, in order to solve this phenomenon, it is necessary to design a reproducible system to ensure that the mRNA can be continuously produced in the host cell. After the host cell is infected with the adenoviral vector, the carried genes are injected into the host cell, and these genes can synthesize mRNAs by utilizing respective base pairs of the host cell. More importantly, the adenovirus is a DNA virus, and the gene amplification ability of the DNA virus in the host is much higher than that of a RNA virus. Therefore, using the adenovirus as a vector will greatly increase the copy number of the mRNA.

After the host cell is infected with the adenoviral vector vaccine, the copy number of the target gene is determined by the strength of the promoter of the adenoviral vector itself, and especially for the replication-defective-type adenoviral vector, its replication function is still relatively weak. In order to solve this problem, in the present invention, the inventor modifies the adenovirus gene to realize the combined action of the promoter of the adenoviral vector itself and the modifying gene, thereby greatly improving the expression rate of the target antigen. The DNA of an adjuvant can also be added into the adenoviral vector carrying the mRNA gene. The adenoviral vector system constructed in this way can not only improve the immunogenicity of the body, but also produce the effect of the adjuvant.

### SUMMARY

The present invention provides an SamRNA vaccine, including a recombinant viral vector which includes: i) a viral gene replication complex including nucleotide sequences encoding viral gene replication-related proteins nsP1, nsP2, nsP3 and nsP4; and ii) a nucleotide sequence encoding at least one antigen.

Preferably, the SamRNA vaccine further includes: iii) a promoter for transcribing an antigen gene.

Preferably, the recombinant viral vector is a recombinant adenovirus, a chimpanzee adenovirus, a recombinant vesicular stomatitis virus, a recombinant poxvirus, a recombinant dengue virus, a recombinant Kunjin virus, a recombinant sendai virus, or a recombinant canine distemper virus. More preferably, the recombinant viral vector is a recombinant adenoviral vector and a chimpanzee adenoviral vector, and more preferably, the adenovirus can be any one of Ad1-Ad52.

The antigen of the present invention causes an immune response against bacteria, viruses, fungi or parasites.

In one embodiment of the present invention, the antigen is a human herpes zoster virus gE protein, a rotavirus VP4 or VP7, an HPV-L1 protein, or a Ebola virus gP protein.

The antigen of the present invention can also be a tumor-specific antigen. For example, the tumor-specific antigen is selected from NY-ESO-1, SSX2, SCP1, RAGE, BAGE, GAGE, MAGE family polypeptides, p53, p21/Ras, CDK4, MUM1, caspase-8, CIA0205, HLA-A2-R1701, β-catenin, TCR, BCR-abl, triosephosphate isomerase, KIA0205, CDC-27, LDLR-FUT, Galectin 4, Galectin 9, protease 3, WT 1, carbonic anhydrase, aldolase A, PRAME, HER-2/neu, mammaglobin, alpha-fetal protein, KSA, gastrin, telomerase catalytic protein, MUC-1, G-250, p53, carcino-embryonic antigens, melanoma-melanocyte differentiation antigens, PAP, PSA, PSMA, PSH-P1, PSM-P1, PSM-P2, p15, Hom/Mel-40, H-Ras, E2A-PRL, H4-RET, IGH-IGK, MYL-RAR, EB viral antigens, EBNA, human papillomavirus antigens, hepatitis B and C viral antigens, human T-lymphotropic viral antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791Tgp72, β-HCG, BCA225, BTAA, CA 125, CA 15-3(CA 27.29\BCAA), CA 195, CA 242, CA-50, CAM43, CD68\KP1, CO-029, FGF-5, Ga733(EpCAM), HTgp-175, M344, MA-50, MG7-Ag, MOV18, NB/70K, NY-CO-1, RCAS1, SDCCAG16, TAAL6, TAG72, TLP, and TPS.

Preferably, the recombinant viral vector is obtained by co-transfecting a modified adenoviral skeleton plasmid and a shuttle plasmid containing an antigen gene. The modified adenoviral skeleton plasmid includes a viral gene replication complex, which is derived from proteins related to replication of coding virus genes of RNA viruses, and more preferably, the viral gene replication complex is nucleotide sequences of nsP1, nsP2, nsP3 and nsP4 from alphaviruses. The adenoviral skeleton plasmid is selected from pAdEasy-1, pAdEasy-2, pBHG11, pBHG-fiber5, or pBHG-fiber35.

In one embodiment of the present invention, the recombinant viral vector further includes: iv) a nucleotide sequence encoding at least one adjuvant. For example, the nucleotide sequence encoding the at least one adjuvant is selected from GM-CSF, IL-17, IFNg, IL-15, IL-21, anti-PD½, lactoferrin, protamine, IL-1, IL-2, IL-3, IL-4, IL-5, IL-6, IL-7, IL-8, IL-9, IL-10, IL-12, INF-α, INF-γ, Lymphotoxin-α, and hGH.

The present invention provides a method for preparing an SamRNA vaccine, including the steps of: constructing a modified adenoviral skeleton plasmid; cloning an antigen gene fragment; constructing a shuttle plasmid; and co-transfecting and packaging the shuttle plasmid and the adenoviral skeleton plasmid.

Preferably, the step of constructing the modified adenoviral skeleton plasmid includes cloning the viral gene replication complex and the promoter, and connecting the viral gene replication complex and the promoter with the adenoviral skeleton plasmid.

Preferably, the adenoviral skeleton plasmid includes a viral gene replication complex, which is gene sequences encoding viral gene replication-related proteins nsP1, nsP2, nsP3 and nsP4. The amino acid sequences of the viral gene replication-related proteins nsP1, nsP2, nsP3 and nsP4 are shown in SEQ ID NO.1, SEQ ID NO.2, SEQ ID NO.3 and SEQ ID NO.4.

Preferably, the adenoviral skeleton plasmid further includes a promoter which is the promoter for transcribing an antigen gene, and a sequence of the promoter is different due to the change of the nucleotide sequence encoding at least one antigen. Preferably, in the present invention, a gene sequence of the promoter is as shown in SEQ ID NO.5.

The adenoviral skeleton plasmid is selected from pAdEasy-1, pAdEasy-2, pBHG11, pBHG-fiber5, pBHG-fiber35, etc.

Preferably, a construction method of a shuttle plasmid containing an antigen gene adopts a conventional experimental method in the industry, which is generally adopted in the art, for example, a nucleotide sequence encoding at least one antigen is synthesized, wherein the same enzymes as those used for the shuttle plasmid are designed to be introduced at both terminals of the synthesized nucleotide sequence encoding at least one antigen, the target antigen and the shuttle plasmid are subjected to double enzyme digestion respectively, then the nucleotide fragments are recovered, and the nucleotide sequence encoding the at least one antigen is connected with the plasmid through a T4 DNA ligase. Specific reference can be made to the method of introducing the viral gene replication complex and the promoter into the skeleton plasmid.

According to the method for preparing the SamRNA vaccine provided by the present invention, a SamRNA vaccine capable of preventing this disease can be developed as long as any antigen gene (i.e., the nucleotide sequence encoding at least one antigen) is inserted into the shuttle plasmid. The vaccine is obtained by co-transfection and recombination of the aforementioned adenoviral skeleton plasmid in which the genes of the viral gene replication complex and the promoter are inserted and the shuttle plasmid containing the antigen gene. The vaccine includes the recombinant viral vector, which includes: i) a viral gene replication complex, which includes nucleotide sequences encoding viral gene replication-related proteins nsP1, nsP2, nsP3 and nsP4, and ii) a nucleotide sequence encoding at least one antigen.

Preferably, the antigen gene is a DNA sequence that can encode any antigen, and any antigen gene that has been discovered or publicly reported can be recombined into an adenoviral vector through the shuttle plasmid, and after the skeleton plasmid and the shuttle plasmid are co-transfected, the SamRNA vaccine of the present invention can be obtained.

Preferably, the antigen gene is selected from a DNA sequence encoding a human herpes zoster virus gE protein, a DNA sequence of a rotavirus VP4 or VP7, a DNA sequence of an HPV-L1 protein, and a DNA sequence of an Ebola virus gP protein, etc.

The shuttle vector is selected from pDC311, pDC312, pDC315, pDC316, p-Shuttle, p-Shuttle-CMV, pAdTrack, pAdTrack-CMV, etc.

The present invention provides a pharmaceutical composition including the SamRNA vaccine, and a pharmaceutically-acceptable adjuvant.

The pharmaceutically-acceptable adjuvant includes a diluent, a solubilizer, an adhesive, a lubricant, a suspending agent, etc.

The dosage form of the pharmaceutical composition includes but is not limited to a freeze-dried preparation, liquid preparation, an emulsion, etc.

Preferably, the pharmaceutical composition is a freeze-dried preparation, and the auxiliary materials of the freeze-dried preparation include mannitol, sucrose, human albumin, and a PB buffer (for maintaining pH of the preparation). In an embodiment of the present invention, in the pharmaceutical composition, the concentration of the mannitol is 10-500 mg/ml, the concentration of the sucrose is 10-500 mg/ml, the concentration of the human albumin is 25-100 mg/ml, and the concentration of the PB buffer is 1-100 mM.

Preferably, the SamRNA vaccine is a liquid injection, and the auxiliary materials added in the liquid preparation include human albumin and the PB buffer. The concentration of the human albumin is 25-100 mg/ml, and the concentration of the PB buffer is 1-100 mM.

The present invention provides a modified adenoviral skeleton plasmid in which a gene of a viral gene replication complex is inserted, wherein the viral gene replication complex is gene sequences encoding viral gene replication-related proteins nsP1, nsP2, nsP3 and nsP4. The gene sequences are inserted into the adenoviral skeleton plasmid in an nsp1-nsp2-nsp3-nsp4 connection manner, and one or more linkers are added between two adjacent protein genes. The linkers include but are not limited to G4S, an LE linker 1 (with a sequence of TTAGAA), an LE linker 2 (with a sequence of CTCGAA), or DEL (with a sequence of GATGAACTG). The amino acid sequences encoding the viral gene replication-related proteins nsP1, nsP2, nsP3 and nsP4 are SEQ ID NO.1, SEQ ID NO.2, SEQ ID NO.3 and SEQ ID NO.4, respectively.

Preferably, the adenoviral skeleton plasmid further includes a promoter which is a promoter for transcribing an antigen gene, and a sequence of the promoter varies with the change of the antigen gene; the promoter can be derived from a promoter when a non-structural protein of a RNA virus is replicated, and can also be derived from common promoters on various expression vectors. Preferably, in the present invention, a gene sequence of the promoter is as shown in SEQ ID NO.5.

Preferably, the promoter and the replication complex genes on the adenoviral skeleton plasmid are codon optimized for a purpose of improving the expression of the replication complex in a host cell, thereby promoting transcription to form more copies of the mRNA. The amino acid sequences of nsP1, nsP2, nsP3 and nsP4 are SEQ ID NO.1, SEQ ID NO.2, SEQ ID NO.3 and SEQ ID NO.4, respectively. The gene sequence of the promoter is as shown in SEQ ID NO.5.

The sequences of nsP1, nsP2, nsP3, nsP4 and the promoter adopt a gene synthesis method, which is known to those of ordinary skills in the art. nsP1, nsP2, nsP3 and nsP4 are directly synthesized into one fragment, and one or more linkers are added between adjacent genes. Enzymatic cleavage sites are added to both terminals of the synthesized sequence, so that the gene fragment can be inserted into the adenoviral skeleton plasmid simply by a T4 DNA ligase.

The adenoviral skeleton plasmid is selected from pAdEasy-1, pAdEasy-2, pBHG11, pBHG-fiber5, and pBHG-fiber35.

The present invention provides a method for preparing the modified adenoviral skeleton plasmid, including the steps of:

cloning the genes of the viral gene replication complex and the promoter, wherein the amino acid sequences of nsP1, nsP2, nsP3 and nsP4 are SEQ ID NO.1, SEQ ID NO.2, SEQ ID NO.3 and SEQ ID NO.4, respectively. The gene sequence of the promoter is as shown in SEQ ID NO.5.

The conventional technology mastered in this art can provide the synthesis business of this gene; and gene synthesis is conducted by adopting conventional experimental techniques in the art. Enzymatic cleavage sites are added to both terminals of the synthesized promoter gene fragment.

Preferably, the amino acid sequences of the viral gene replication complexes nsP1, nsP2, nsP3 and nsP4 are SEQ ID NO.1, SEQ ID NO.2, SEQ ID NO.3 and SEQ ID NO.4. The four protein genes are synthesized into one gene fragment by adopting the order of nsP1-linker-nsP2-linker-nsP3-linker-nsP4, and one or more linkers are added between two protein genes. The linkers include but are not limited to G4S, an LE linker 1 (with a nucleotide sequence of TTAGAA), an LE linker 2 (with a nucleotide sequence of CTCGAA), or DEL (with a nucleotide sequence of GATGAACTG). Enzymatic cleavage sites are added to both sides of the gene fragment.

Insertion of the gene fragments of the viral gene replication complex and the promoter into the adenoviral skeleton plasmid: the connection of the gene fragments of the viral gene replication complex and the promoter with the adenoviral skeleton plasmid is conducted by adopting a conventional experimental method in the industry, and those of ordinary skills in molecular biology in the art can carry out this operation.

Preferably, the gene fragment of the viral gene replication complex is connected with the adenoviral skeleton plasmid, and the specific operation is that the adenoviral skeleton plasmid and the viral gene replication complex are respectively subjected to double enzyme digestion at 37° C.; after the enzyme digestion is completed, a target gene fragment is recovered through gel extraction, the recovery of the target gene fragment is conducted by adopting a gel extraction kit, and the specific operation can be carried out according to the instructions of the kit. Then, the gene fragment of the viral gene replication complex and the adenoviral skeleton plasmid which are subjected to double enzyme digestion are ligated by a DNA ligation kit/T4DNA ligase, and the ligation is conducted overnight. Thereafter, the ligation product is transformed into DH5a competent cells. An LB medium is inoculated with clones identified as positive by PCR, and the clones are kept at 37° C. at 200-2250 rpm overnight. On the next day, the bacterial cells are recovered, and the plasmid is recovered by a plasmid extraction kit.

Preferably, the gene fragment of the promoter is inserted into the adenoviral skeleton plasmid to which the fragment of the viral gene replication complex has already been ligated. The method for inserting the gene fragment of the promoter into the adenoviral skeleton plasmid is similar to the aforementioned method. The adenoviral skeleton plasmid to which the viral gene replication complex has already been ligated, and the promoter, are subjected to double enzyme digestion at 37° C.; after the enzyme digestion is completed, a target gene fragment is recovered through gel extraction, the recovery of the target gene fragment is conducted by adopting a gel extraction kit, and the specific operation can be carried out according to the instructions of the kit. Then, the gene fragment of the viral gene replication complex and the adenoviral skeleton plasmid which are subjected to double enzyme digestion are ligated by a DNA ligation kit/T4DNA ligase, and the ligation is conducted overnight. Thereafter, the ligation product is transformed into DH5a competent cells. An LB medium is inoculated with clones identified as positive by PCR, and the clones are kept at 37° C. at 200-2250 rpm overnight. On the next day, the bacterial cells are recovered, and the plasmid is recovered by a plasmid extraction kit.

Preferably, the constructed adenoviral skeleton plasmid is identified by double enzyme digestion and PCR methods.

The present invention solves the risk that the mRNA vaccine is greatly degraded in vivo; although the existing encapsulating technology can avoid the significant degradation of the mRNA vaccine, the defect that it is difficult for the mRNA to directly enter cells (low bioavailability) cannot be solved. The samRNA vaccine of the present invention is an adenoviral vector carrying an antigen, and the adenoviral vector can directly infect human and animal cells, thereby injecting the target antigen gene into the host cell. The conventional mRNA vaccine can only be translated once after entering the host cell, while the samRNA vaccine of the present invention can use RNA as a template to synthesize a large amount of mRNAs, thereby greatly increasing the expression quantity of the antigen.

A common replication-defective-type adenoviral vector can only replicate in cells like HEK293, and can only infect and cannot replicate for humans and animals, so its replication ability in organisms is limited. However, after the adenoviral vector vaccine in which the viral gene replication complex and the promoter are inserted, provided by the present invention, infects the host cell, nucleic acid is injected into the host cell. Then a series of dynamic reactions will take place in the host cell. Firstly, the transcription of the gene of the viral gene replication complex and the translation of a protein occur. Meanwhile, the antigen gene is transcribed into mRNA. With the translation of the proteins nsP1, nsP2, nsP3 and nsP4, the four proteins will form a viral gene replication complex of a viral genome. Then, under the action of the viral gene replication complex, a large number of mRNAs are synthesized by taking an antigen gene RNA as a template, and these mRNAs use various proteases of the host cell to synthesize the target antigens. The process in which the viral gene replication complex takes the RNA as a template to synthesize mRNA, is a continuous process, and antigen expression lasts longer, so with the modified adenoviral vector, the action of the SamRNA vaccine lasts longer.

The literature reports that a virus is used as a vector of SamRNA, but the shortcomings are obvious. First of all, the viruses used for samRNA are all RNA viruses, and are positive-sense RNA viruses, and these viruses have limited infection and replication abilities. Host restriction makes it very difficult to produce SamRNA using the virus as the vector, and strict host cell restriction makes it difficult for a conventional production process to meet the production of such a virus. In addition, the limited amplification capacity further seriously affects the yield of the virus. In addition, for the SamRNA using the virus as the vector, the outer-membrane structure gene of the RNA virus is replaced by the antigen gene in the process of genome recombination and construction. The deletion of the outer-membrane structure gene of the virus has the advantage that no virus will be assembled after animal and human host cells are infected with the virus, thereby reducing the risk of infection or pathopoiesia. However, the shortcomings are obvious. The depletion of the structural gene causes the virus vector vaccine to be unable to complete the assembly of virus particles under conventional conditions, and the technical difficulty of commercial production is relatively high.

### BRIEF DESCRIPTION OF THE DRAWINGS

FIG. 1 shows the identification results of inserting a viral gene replication complex protein gene into a skeleton plasmid;

FIG. 2 shows the identification results of inserting a promoter sequence into the skeleton plasmid;

FIG. 3 shows a modified skeleton plasmid;

FIG. 4 shows a flow diagram of constructing Ad-SamRNA-gE;

FIG. 5 shows the double enzyme digestion verification of Ad-SamRNA-gE;

FIG. 6 shows a chromatogram of Ad-SamRNA-gE purified by CL-4B;

FIG. 7 shows the purity analysis of Ad-SamRNA-gE;

FIG. 8 shows the immunogenicity of different vectors, wherein the left side is the immunogenicity of primary immunization, and the right side is the immunogenicity of secondary immunization;

FIG. 9 shows the expression quantities of gE genes of different vectors; and

FIG. 10 shows that the molecular adjuvant enhances the immunogenicity of Ad-SamRNA-gE, wherein the left side is the immunogenicity of primary immunization, and the right side is the immunogenicity of secondary immunization.

### DETAILED DESCRIPTION

The technical solutions in the embodiments of the present invention will be clearly and completely described below. Apparently, the described embodiments are merely a part of embodiments rather than all embodiments of the present invention. All other embodiments obtained by a person of ordinary skill in the art based on the embodiments of the present invention without creative efforts shall fall within the protection scope of the present invention.

### Embodiment 1 Insertion of Viral Gene Replication Complex and Promoter Gene

Fragments into a Skeleton Plasmid

Synthesis of viral gene replication complex and promoter gene fragments: In a specific implementation, the gene sequences of the viral gene replication complexes nsP1, nsP2, nsP3 and nsP4 were SEQ ID NO.6, SEQ ID NO.7, SEQ ID NO.8 and SEQ ID NO.9, respectively, and the viral gene replication complex gene fragment is synthesized according to the aforementioned sequences, wherein four antigen protein genes were synthesized into a large gene fragment.

For the promoter, a gene fragment of the promoter was synthesized according to SEQ ID NO.5, and meanwhile, enzymatic cleavage sites were added at both terminals of the gene fragment of the promoter.

Taking a pAdEasy-1 adenoviral skeleton plasmid as an example, a process of inserting the viral gene replication complex and the promoter into pAdEasy-1 was presented. Firstly, the gene fragment of the viral gene replication complex was inserted into the pAdEasy-1 adenoviral skeleton plasmid, wherein the 4 synthesized long protein gene fragments of the viral gene replication complex contained pad enzymatic cleavage sites on both sides. pAdEasy-1 and the 4 protein genes of the viral gene replication complex were respectively digested with a pac I enzyme. After gel extraction, ligation was conducted by a T4 DNA ligase. Theoretically, there were two ligation manners of the protein genes of the viral gene replication complex (insertion into the skeleton plasmid in an inverted order and insertion into the skeleton plasmid in a sequential order). After the ligation product was transformed into DH5a, monoclones were selected, and the vector in which the genes were inserted into the skeleton plasmid as expected was identified by a PCR method. The results of PCR identification were shown in FIG. 1. The clone No. 1 and the clone No. 2 were both correctly inserted into the protein gene fragment of the viral gene replication complex, and the clone No. 1 was selected to be subjected to an operation of inserting the gene fragment of the promoter.

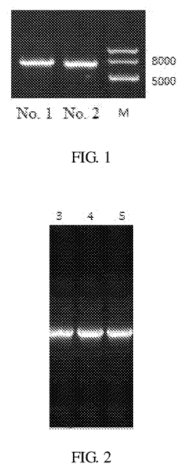

The gene of the promoter and the skeleton plasmid into which the gene of the viral gene replication complex has been inserted, were digested with ClaI, and then ligated with a T4 DNA ligase after gel extraction. Theoretically, there were two ligation manners of the protein genes of the promoter (insertion into the skeleton plasmid in an inverted order and insertion into the skeleton plasmid in a sequential order). After the ligation product was transformed into DH5a, monoclones were selected, and the vector in which the genes were inserted into the skeleton plasmid as expected was identified by a PCR method. The results of PCR identification were shown in FIG. 2. In this step of operation, clones No. 3, No. 4 and No. 5 were correctly inserted into the promoter sequence.

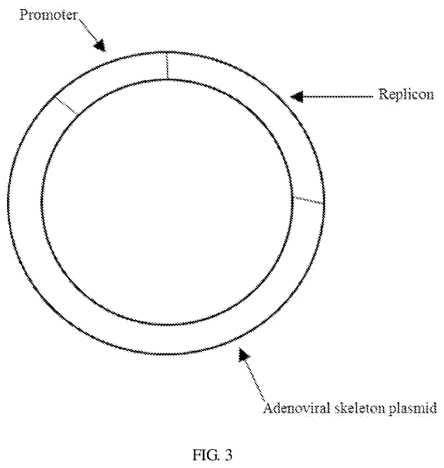

After the aforementioned two steps, the skeleton plasmids pAdEasy-No. 13, No. 4 and No. 5 with addition of the sequences of the viral gene replication complex and the promoter, will be obtained. The positions of the promoter and the viral gene replication complex in the pAdEasy-1 skeleton plasmid were shown in FIG. 3.

According to the same method, the insertion of sequences of the promoter and the viral gene replication complex could be completed on other skeleton plasmids.

### Embodiment 2 Construction of Ad-SamRNA-gE

Taking the herpes zoster gE protein antigen as an example, the display of a construction process of an Ad (adenovirus)-SamRNA vaccine was conducted.

A gE antigen gene was synthesized, and meanwhile enzymatic cleavage sites were added on both sides of the gene fragment, the specific enzymatic cleavage sites were determined according to the selected recombination system and shuttle plasmid, and this method was mastered by those skilled in the art. The Adeasy vector system was taken as an example to display the construction process of the Ad-SamRNA vaccine hereafter.

Firstly, the gE antigen gene was ligated into the shuttle plasmid pS-C to form pS-C-gE, and the ligation method adopted an experimental method commonly used in the industry. The shuttle plasmid pS-C and the gE antigen gene were subjected to double enzyme digestion with KpnI and XhoI at the same time (see Table 1 below for the enzyme digestion reaction system). After the enzyme digestion reaction was completed, the target fragments were recovered with a gel extraction kit (see the instructions for the recovery method). Then the linearized antigen fragment and the plasmid were ligated by a T4 DNA ligase (see Table 2 below for the reaction system).

The construction methods adopting other adenoviral vector systems were similar to the above.

TABLE 1
Double-enzyme digestion reaction system (50 μl)

| Reagent | Volume |
|---|---|
| Enzyme A | 2 μl |
| Enzyme B | 2 μl |
| Buffer | 5 μl |
| Shuttle plasmid or gE gene fragment | 30 μl |
| water | 11 μl |

The double enzyme digestion reaction was carried out at 37° C. for at least 4 h or above.

TABLE 2
Enzyme ligation reaction system (10 μl)

| Reagent | Volume |
|---|---|
| Shuttle plasmid | 2 μl |
| gE gene fragment | 2 μl |
| Buffer | 1 μl |
| T4 DNA ligase | 1 μl |
| H2O | |

The ligation reaction was carried out at 4° C. overnight.

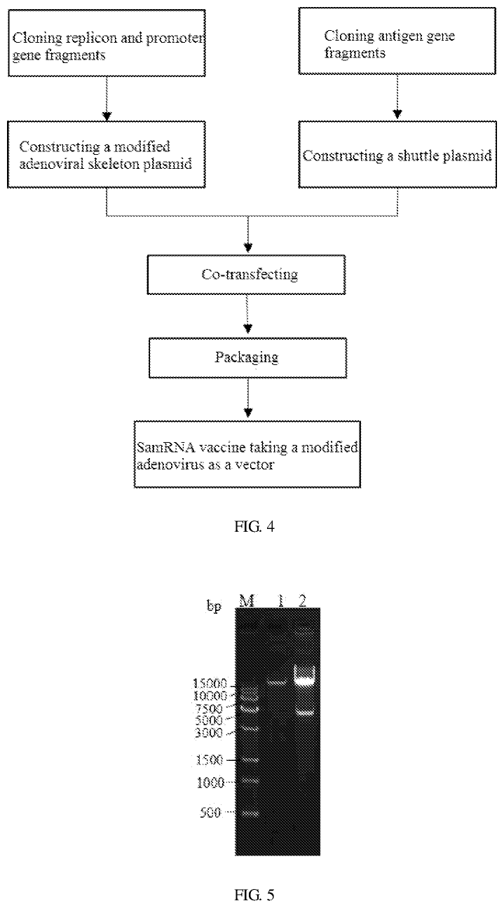

The shuttle plasmid pS-C-gE containing an antigen gene, and the adenoviral skeleton plasmid (such as pAdEasy-1) in which the viral gene replication complex and the promoter were inserted, were used for co-transfection, the two plasmids would undergo homologous recombination, and Ad-SamRNA-gE was obtained through separation. The flow diagram of constructing Ad-SamRNA was shown in FIG. 4.

The constructed Ad-SamRNA-gE was verified by a double enzyme digestion method, and the successfully constructed vector would be used for production and immunogenicity evaluation of the vaccine. The results of double enzyme digestion were as shown in FIG. 5 below: a lane M was a marker, a lane 1 was double enzyme digestion of the adenoviral vector, and a lane 2 was double enzyme digestion of Ad-SamRNA-gE. The results showed that the gE antigen gene was correctly integrated into the adenoviral vector system.

### Embodiment 3 Preparation of Ad-SamRNA-gE Vaccine

Taking the herpes zoster gE protein antigen as an example, the display of a preparation process of an Ad-SamRNA vaccine was conducted.

An adenoviral vector could massively propagate in 293 cells. HEK293 cells were mostly used for the Admax adenoviral vector system, while AD293 cells were used for Adeasy recombinant adenoviral vector cells.

293 cells were infected with Ad-SamRNA-gE at MOI=5-10 for at least 40 h, and then centrifuged at 8,000 g for 10 min to collect cell precipitates. The cell precipitates were dissolved in PB or a lysis buffer (2 mM MgCl₂, 50 mM HEPES, pH 7.5), and then repeatedly frozen and thawed at −80° C. for three times for cell lysis, the cell debris was removed by centrifugation, and the supernatant passed through CL-4B, and subjected to one-step chromatography to obtain a target virus.

Besides cell debris, various impure proteins in the cell lysis solution were Ad-SamRNA-gE, and the cell debris could be removed by centrifugation. Compared with impure proteins, the molecular weight of Ad-SamRNA-gE was much larger than those of impure proteins, and therefore, a pure virus could be obtained by a one-step process with CL-4B.

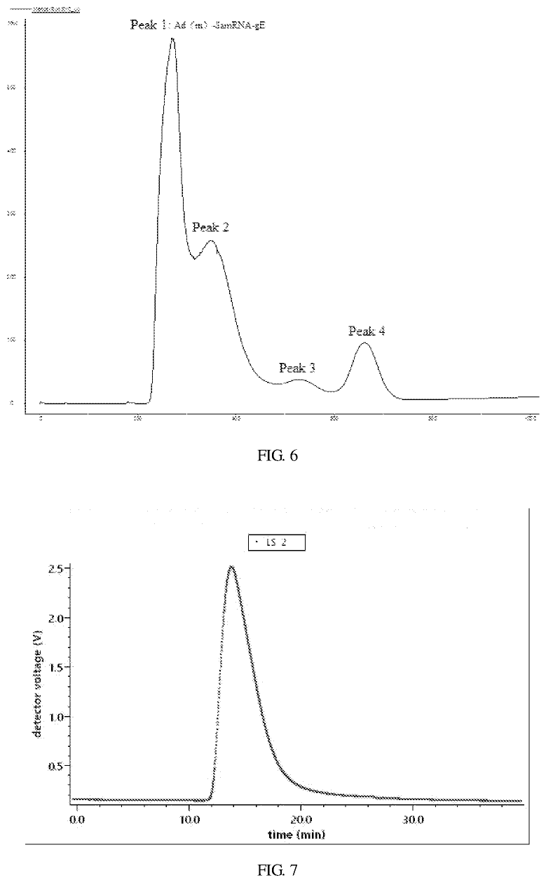

The result of purifying Ad-SamRNA-gE by CL-4B was shown in FIG. 6. The molecular weight of Ad-SamRNA-gE was the largest, and thus was eluted first. The peak 1 in FIG. 6 was Ad-SamRNA-gE.

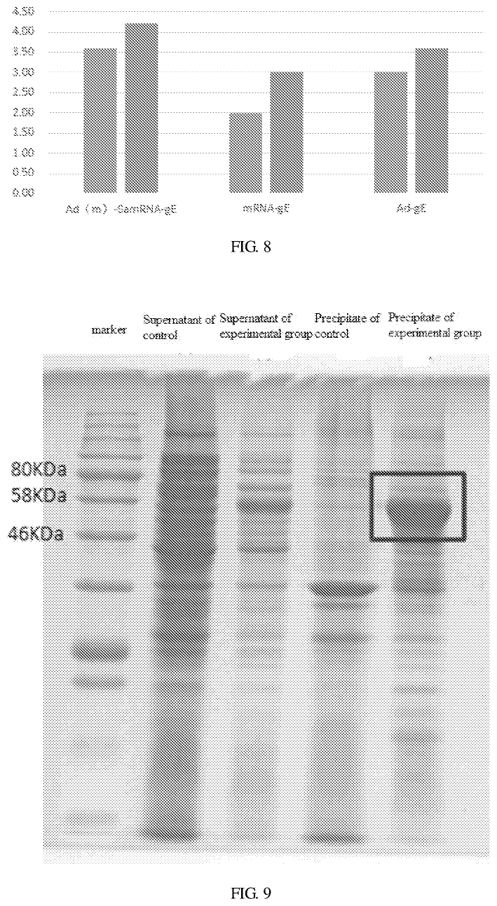

The purity of the harvested Ad-SamRNA-gE was analyzed by HPLC. A TSK5000 column was selected for the experiment. The HPLC result was shown in FIG. 7. It could be seen that no impurity peak could be seen for the harvested virus, and thus the purity of the virus solution was very high.

### Embodiment 4 Study on Immunogenicity of Ad-SamRNA-gE Vaccine

Taking the herpes zoster gE protein antigen as an example, the display of the immunogenicity of the Ad-SamRNA vaccine was conducted.

An experimental group: an Ad-SamRNA-gE group prepared in Embodiment 3;

A control group 1: a group directly injected with mRNA of the gE protein, which was referred to as mRNA-gE for short;

A control group 2: gE protein antigen gene vaccine using a common adenovirus as a vector, referred to as Ad-gE for short;

A negative control group: a normal saline immunization group

Experimental animals: NIH mice were taken, with 10 in each group, and a weight of 12-14 g.

Immunization mode: subcutaneous injection, wherein the experimental group, the control group 1 and the control group 2 were injected with drugs at a single injection dose of 1×10⁸ IFU. Each mouse was immunized with two injections, with an interval of 4 weeks between the two injections. Primary immunization blood was collected from the orbit before the immunization with the second injection, and the eyeball was enucleated to collect the secondary immunization serum 28 days after the last immunization. The antibody titer in the serum was determined by ELISA, and the results were shown in FIG. 8.

It could be seen according to the results shown in FIG. 8 that the immunogenicity of Ad-SamRNA-gE was superior to that of Ad-gE and significantly superior to that of mRNA-gE, with significant difference. Therefore, it was proved that the antigen gene vaccine taking the modified adenovirus as the vector, prepared by the present invention, could greatly improve the immunogenicity of the mRNA vaccine.

### Embodiment 5 Determination of Expression Quantities of Ad-SamRNA-gE and Ad-gE

293 cells were infected with the constructed Ad-SamRNA-gE and Ad-gE at MOI=10, incubated in a 5% CO₂ incubator at 37° C. for 40 h, and then centrifuged to collect a cell supernatant and cell precipitates, and the cell precipitates was lysed with a lysis buffer (2 mM MgCl₂, 50 mM HEPES, pH 7.5); and the expression quantities of the gE protein in the two vectors was analyzed by SDS-PAGE. The results were shown in FIG. 9.

FIG. 9 demonstrated that compared with the control group Ad-gE, the expression quantity of the gE protein was significantly higher after the cells were infected with the sample to be tested Ad-SamRNA-gE. The result corresponded to the case that the immunogenicity of Ad-SamRNA-gE is much higher than that of Ad-gE in Embodiment 4.

### Embodiment 6 Immunogenicity of Molecular Adjuvant Against Ad-SamRNA-gE

Taking the herpes zoster gE protein antigen as an example, the display of the immunogenicity of the Ad-SamRNA vaccine was conducted.

Experimental groups: Ad-SamRNA-gE with C3b and Ad-SamRNA-gE without C3b were prepared respectively.

A negative control group: a normal saline immunization group

Experimental animals: NIH mice were taken, with 10 in each group, and a weight of 12-14 g.

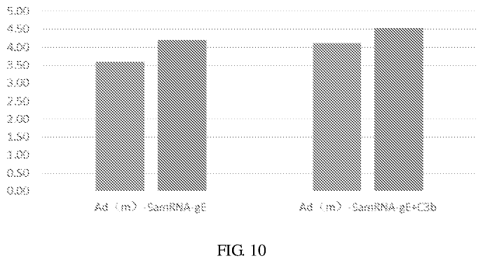

Immunization mode: subcutaneous injection, wherein the experimental group, the control group 1 and the control group 2 were injected with drugs at a single injection dose of 1×10⁸ IFU. Each mouse was immunized with two injections, with an interval of 4 weeks between the two injections. Primary immunization blood was collected from the orbit before the immunization with the second injection, and the eyeball was enucleated to collect the secondary immunization serum 28 days after the last immunization. The antibody titer in the serum was determined by ELISA, and the results were shown in FIG. 10.

According to the results in FIG. 10, C3b could enhance the immunogenicity of Ad-SamRNA-gE.

Finally, it should be noted that the embodiments described above are only illustrative of the technical solutions of the present invention, rather than limiting the present invention; although the present invention is described in detail with reference to the foregoing embodiments, it should be understood by those of ordinary skills in the art that modifications still can be made to the technical solutions described in the foregoing embodiments or equivalent replacements can be made to some or all technical features in the foregoing embodiments; and these modifications and replacements would not make the nature of the corresponding technical solutions depart from the scope of the technical solutions of the embodiments of the present invention.

1. An SamRNA vaccine, comprising a recombinant viral vector which comprises: i) a viral gene replication complex comprising nucleotide sequences encoding viral gene replication-related proteins nsP1, nsP2, nsP3, and nsP4; and ii) a nucleotide sequence encoding at least one antigen.

2. The SamRNA vaccine according to claim 1, wherein the recombinant viral vector is a recombinant adenovirus, a chimpanzee adenovirus, a recombinant vesicular stomatitis virus, a recombinant poxvirus, a recombinant dengue virus, a recombinant Kunjin virus, a recombinant sendai virus, or a recombinant canine distemper virus.

3. The SamRNA vaccine according to claim 1, wherein the antigen causes an immune response against bacteria, viruses, fungi or parasites.

4. The SamRNA vaccine according to claim 3, wherein the antigen is a human herpes zoster virus gE protein, a rotavirus VP4 or VP7, an HPV-L1 protein, or an Ebola virus gP protein.

5. The SamRNA vaccine according to claim 1, wherein the antigen is a tumor-specific antigen, and is selected from NY-ESO-1, SSX2, SCP1, RAGE, BAGE, GAGE, MAGE family polypeptides, p53, p21/Ras, CDK4, MUM1, caspase-8, CIA0205, HLA-A2-R1701, (3-catenin, TCR, BCR-abl, triosephosphate isomerase, KIA0205, CDC-27, LDLR-FUT, Galectin 4, Galectin 9, protease 3, WT 1, carbonic anhydrase, aldolase A, PRAME, HER-2/neu, mammaglobin, alpha-fetal protein, KSA, gastrin, telomerase catalytic protein, MUC-1, G-250, p53, carcino-embryonic antigens, melanoma-melanocyte differentiation antigens, PAP, PSA, PSMA, PSH-P1, PSM-P1, PSM-P2, p15, Hom/Mel-40, H-Ras, E2A-PRL, H4-RET, IGH-IGK, MYL-RAR, EB viral antigens, EBNA, human papillomavirus antigens, hepatitis B and C viral antigens, human T-lymphotropic viral antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791Tgp72, β-HCG, BCA225, BTAA, CA 125, CA 15-3(CA 27.29\BCAA), CA 195, CA 242, CA-50, CAM43, CD68\KP1, CO-029, FGF-5, Ga733(EpCAM), HTgp-175, M344, MA-50, MG7-Ag, MOV18, NB/70K, NY-CO-1, RCAS1, SDCCAG16, TAAL6, TAG72, TLP, and TPS.

6. The SamRNA vaccine according to claim 1, wherein the recombinant viral vector is obtained by co-transfecting a modified adenoviral skeleton plasmid and a shuttle plasmid containing a nucleotide sequence encoding at least one antigen, wherein the modified adenoviral skeleton plasmid comprises a viral gene replication complex which is gene sequences encoding viral gene replication-related proteins nsP1, nsP2, nsP3 and nsP4.

7. The SamRNA vaccine according to claim 6, wherein the adenoviral skeleton plasmid is selected from pAdEasy-1, pAdEasy-2, pBHG11, pBHG-fiber5 or pBHG-fiber35.

8. The SamRNA vaccine according to claim 1, wherein the recombinant viral vector further comprises: iii) a promoter for transcribing an antigen gene.

9. The SamRNA vaccine according to claim 1, wherein the recombinant viral vector further comprises: iv) a nucleotide sequence encoding at least one adjuvant.

10. The SamRNA vaccine according to claim 8, wherein the adjuvant is selected from C3b, GM-CSF, IL-17, IFN, IL-15, IL-21, IL-1, IL-2, IL-3, IL-4, IL-5, IL-6, IL-7, IL-8, IL-9, IL-10, IL-12, INF-α, INF-γ and CpG.

11. A method for preparing the SamRNA vaccine according to claim 1, comprising the steps of: constructing a modified adenoviral skeleton plasmid; cloning an antigen gene fragment; constructing a shuttle plasmid; co-transfecting and packaging the shuttle plasmid and the adenoviral skeleton plasmid, wherein the modified adenoviral skeleton plasmid comprises a viral gene replication complex which is gene sequences encoding viral gene replication-related proteins nsP1, nsP2, nsP3, and nsP4.

12. (canceled)

13. (canceled)

14. A modified adenoviral skeleton plasmid, comprising a viral gene replication complex which is gene sequences encoding viral gene replication-related proteins nsP1, nsP2, nsP3 and nsP4, wherein the adenoviral skeleton plasmid is selected from pAdEasy-1, pAdEasy-2, pBHG11, pBHG-fiber5, or pBHG-fiber35.


SEQUENCE LISTING (9 sequences)

SEQ ID NO: 1 - protein, 537 aa, Artificial Sequence
  Synthetic Peptide
MAAKVHVDIEADSPFIKSLQKAFPSFEVESLQVTPNDHANARAFSHLATKLIEQETDKDTLILDIGSAPSRRMMSTHKYHCVCPMRSAEDPERLDSYAKKLAAASGKVLDREIAGKITDLQTVMATPDAESPTFCLHTDVTCRTAAEVAVYQDVYAVHAPTSLYHQAMKGVRTAYWIGFDTTPFMFDALAGAYPTYATNWADEQVLQARNIGLCAASLTEGRLGKLSILRKKQLKPCDTVMFSVGSTLYTESRKLLRSWHLPSVFHLKGKQSFTCRCDTIVSCEGYVVKKITMCPGLYGKTVGYAVTYHAEGFLVCKTTDTVKGERVSFPVCTYVPSTICDQMTGILATDVTPEDAQKLLVGLNQRIVVNGRTQRNTNTMKNYLLPIVAVAFSKWAREYKADLDDEKPLGVRERSLTCCCLWAFKTRKMHTMYKKPDTQTIVKVPSEFNSFVIPSLWSTGLAIPVRSRIKMLLAKKTKRELIPVLDASSARDAEQEEKERLEAELTREALPPLVPIAPAETGVVDVDVEELEYHAGA

SEQ ID NO: 2 - protein, 798 aa, Artificial Sequence
  Synthetic Peptide
GVVETPRSALKVTAQPNDVLLGNYVVLSPQTVLKSSKLAPVHPLAEQVKIITHNGRAGGYQVDGYDGRVLLPCGSAIPVPEFQALSESATMVYNEREFVNRKLYHIAVHGPSLNTDEENYEKVRAERTDAEYVFDVDKKCCVKREEASGLVLVGELTNPPFHEFAYEGLKIRPSAPYKTTVVGVFGVPGSGKSAIIKSLVTKHDLVTSGKKENCQEIVNDVKKHRGKGTSRENSDSILLNGCRRAVDILYVDEAFACHSGTLLALIALVKPRSKVVLCGDPKQCGFFNMMQLKVNFNHNICTEVCHKSISRRCTRPVTAIVSTLHYGGKMRTTNPCNKPIIIDTTGQTKPKPGDIVLTCFRGWAKQLQLDYRGHEVMTAAASQGLTRKGVYAVRQKVNENPLYAPASEHVNVLLTRTEDRLVWKTLAGDPWIKVLSNIPQGNFTATLEEWQEEHDKIMKVIEGPAAPVDAFQNKANVCWAKSLVPVLDTAGIRLTAEEWSTIITAFKEDRAYSPVVALNEICTKYYGVDLDSGLFSAPKVSLYYENNHWDNRPGGRMYGFNAATAARLEARHTFLKGQWHTGKQAVIAERKIQPLSVLDNVIPINRRLPHALVAEYKTVKGSRVEWLVNKVRGYHVLLVSEYNLALPRRRVTWLSPLNVTGADRCYDLSLGLPADAGRFDLVFVNIHTEFRIHHYQQCVDHAMKLQMLGGDALRLLKPGGILMRAYGYADKISEAVVSSLSRKFSSARVLRPDCVTSNTEVFLLFSNFDNGKRPSTLHQMNTKLSAVYAGEAMHTAGC

SEQ ID NO: 3 - protein, 482 aa, Artificial Sequence
  Synthetic Peptide
APSYRVKRADIATCTEAAVVNAANARGTVGDGVCRAVAKKWPSAFKGAATPVGTIKTVMCGSYPVIHAVAPNFSATTEAEGDRELAAVYRAVAAEVNRLSLSSVAIPLLSTGVFSGGRDRLQQSLNHLFTAMDATDADVTIYCRDKSWEKKIQEAIDMRTAVELLNDDVELTTDLVRVHPDSSLVGRKGYSTTDGSLYSYFEGTKFNQAAIDMAEILTLWPRLQEANERICLYALGETMDNIGSKCPVNDSDSSTPPRTVPCLCRYAMTAERIARLRSHQVKSMVVCSSFPLPKYHVDGVQKVKCEKVLLFDPTVPSVVSPRKYAASTTDHSDRSLRGFDLDWTTDSSSTASDTMSLPSLQSCDIDSIYEPMAPIVVTADVHPEPAGIADLAADVHPEPADHVDLENPIPPPRPKRAAYLASRAAERPVPAPRKPTPAPRTAFRNKLPLTFGDFDEHEVDALASGITFGDFDDVLRLGRAGA

SEQ ID NO: 4 - protein, 614 aa, Artificial Sequence
  Synthetic Peptide
YIFSSDTGSGHLQQKSVRQHNLQCAQLDAVQEEKMYPPKLDTEREKLLLLKMQMHPSEANKSRYQSRKVENMKATVVDRLTSGARLYTGADVGRIPTYAVRYPRPVYSPTVIERFSSPDVAIAACNEYLSRNYPTVASYQITDEYDAYLDMVDGSDSCLDRATFCPAKLRCYPKHHAYHQPTVRSAVPSPFQNTLQNVLAAATKRNCNVTQMRELPTMDSAVFNVECFKRYACSGEYWEEYAKQPIRITTENITTYVTKLKGPKAAALFAKTHNLVPLQEVPMDRFTVDMKRDVKVTPGTKHTEERPKVQVIQAAEPLATAYLCGIHRELVRRLNAVLRPNVHTLFDMSAEDFDAIIASHFHPGDPVLETDIASFDKSQDDSLALTGLMILEDLGVDQYLLDLIEAAFGEISSCHLPTGTRFKFGAMMKSGMFLTLFINTVLNITIASRVLEQRLTDSACAAFIGDDNIVHGVISDKLMAERCASWVNMEVKIIDAVMGEKPPYFCGGFIVFDSVTQTACRVSDPLKRLFKLGKPLTAEDKQDEDRRRALSDEVSKWFRTGLGAELEVALTSRYEVEGCKSILIAMTTLARDIKAFKKLRGPVIHLYGGPRLVR

SEQ ID NO: 5 - DNA, 193 bp, Artificial Sequence
  Synthetic Construct
gagtggacctgtgtcgcgtaaatctaaaagtaagagtgaggcagaatctttttcggatagtggcgcttctgagccactaagttcataatcaagatgtcttactctacttctggtttgcgttctttgcctgcatatactaagtctttttgtccttattatgctttgtatgatctgttggtgtcagcccaaggtg

SEQ ID NO: 6 - DNA, 1614 bp, Artificial Sequence
  Synthetic Construct
atggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcataa

SEQ ID NO: 7 - DNA, 2400 bp, Artificial Sequence
  Synthetic Construct
atgggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgcttgccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgttaa

SEQ ID NO: 8 - DNA, 1452 bp, Artificial Sequence
  Synthetic Construct
atggcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaggaggcaaacgaacggatatgcctatacgcgctgggcgaaacaatggacaacatcggatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcataa

SEQ ID NO: 9 - DNA, 1908 bp, Artificial Sequence
  Synthetic Construct
atgtatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatgaccaccttggcgagggacattaaggcgtttaagaaattgagaggacctgttatacacctctacggcggtcctagattggtgcgttaatacacagaattctgattatagcgcactattatagcaccatgaattacatccctacgctaa